(19) United States
(12) Patent Application Publication
(10) Pub. No.: US 2005/0049433 A1
(43) Pub. Date: Mar. 3, 2005

(54) Recovery of carboxylic acid from byproduct stream

(75) Inventors: Maohong Fan, Ames, IA (US); Robert C. Brown, Ames, IA (US)

Correspondence Address: MCKEE, VOORHEES & SEASE, P.L.C., 801 GRAND AVENUE, SUITE 3200, DES MOINES, IA, 50309-2721, US

(73) Assignee: Iowa State University Research Foundation, Ames, IA (US)

(21) Appl. No.: 10/927,364

(22) Filed: Aug. 26, 2004

Related U.S. Application Data
(60) Provisional application No. 60/498,053, filed on Aug. 27, 2003.

(51) Int. Cl. C07C 51/42

(52) U.S. Cl. 562/513

(57) ABSTRACT

A novel method for recovering carboxylic acid from a waste water stream is described. The method involves first neutralizing the carboxylic acid in the waste water with CaO or Ca(OH)₂ to form calcium carboxylate. The resulting calcium carboxylate is then reacted with sulfite or sulfate to regenerate the carboxylic acid. The resulting waste water typically has a carboxylic acid content of at least 25-wt %.

### CROSS-REFERENCE TO RELATED APPLICATIONS

This application is a conversion of U.S. Provisional Application No. 60/498,053 filed Aug. 27, 2003, which is herein incorporated by reference in its entirety.

### FIELD OF THE INVENTION

This invention relates to the recovery of carboxylic acids from a byproduct stream.

### BACKGROUND OF THE INVENTION

The utilization of waste materials or byproducts of agriculture has long been investigated as a potential solution to waste management, energy, and chemical supply problems. Typically, chemicals such as alcohols are produced by enzymatically converting treated agriculture waste materials or byproducts with extracellular enzymes that hydrolyze polysaccharides into soluble sugars, which are then fermented into ethanol and normally recovered by distillation.

Acetic acid is an important industrial chemical, about 7.835 billion pounds of which was produced in the United States in 2002. As one of the most widely used carboxylic acids, it is often used as a raw material to prepare other valuable products such as acetic esters. Another important application of acetic acid is to serve as a solvent to facilitate many industrial processes, such as the manufacture of cellulose acetate and pharmaceutical products. There is therefore considerable interest in producing acetic acid from agricultural materials.

Acetic acid produced today is primarily based on natural gas. However, as a nonrenewable resource, and at current high rates of consumption, natural gas is barely able to support the acetic acid industry. It is anticipated that, unless other production methods are successfully developed, the price of acetic acid will increase markedly in the future. As a promising alternative, the production of acetic acid using biomass materials has gained more interest, primarily due to it cost-effectiveness. However, the main obstacle to widespread use of this method is the difficulty of separating acetic acid from an anaerobic system or waste streams from biological processes. To alleviate the negative effects of acid accumulation, the generated acid must be either removed or neutralized.

The recovery of acetic acid from dilute aqueous solutions is difficult and costly. Some conventional methods of recovery are simple distillation and azeotropic distillation. However, the cost of these methods is too high for commercial applications unless the concentration of acetic acid is large, i.e. in excess of 20 wt %. The typical concentration of acetic acid in waste streams from the agriculture industry is typically only a few percent. In the case of anaerobic digestion, it is less than 0.5 wt %.

### SUMMARY OF THE INVENTION

The present invention relates to a method of separating acetic acid and other carboxylic acids from industrial waste streams. The method involves first combining the carboxylic acid containing waste stream with a calcium oxide or calcium hydroxide source to form a calcium carboxylate. Carboxylic acid is then regenerated through reaction with a sulfur oxide. Calcium sulfite or sulfate produced in this same reaction may then be recycled by decomposition into sulfur dioxide or sulfur trioxide and calcium oxide for reuse in the overall recovery process.

The invention provides several advantages over conventional carboxylic acid recovery procedures. First, the method is less expensive because it does not require the use of organic chemicals and can be conducted at comparatively lower temperatures. It is also safer for the environment since it requires less energy, and allows for recycling of the by-products from the separation procedure. Further, the method separates carboxylic acid with greater efficiency than previously available separation methods.

### DETAILED DESCRIPTION OF THE DRAWINGS

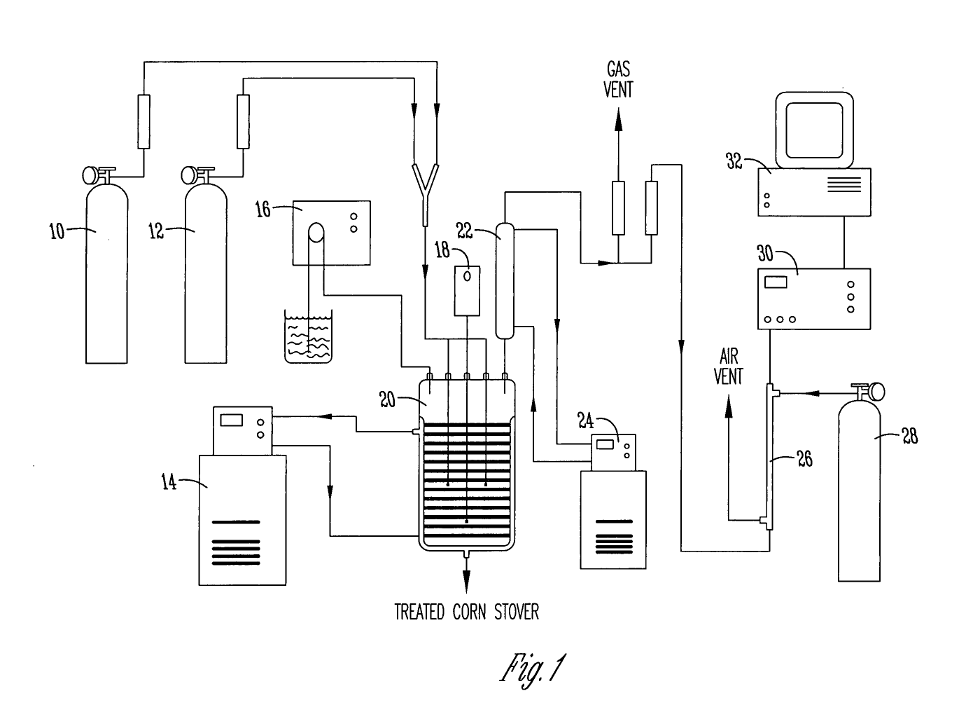

FIG. 1 is an apparatus for the regeneration of carboxylic acid using SOₓ.

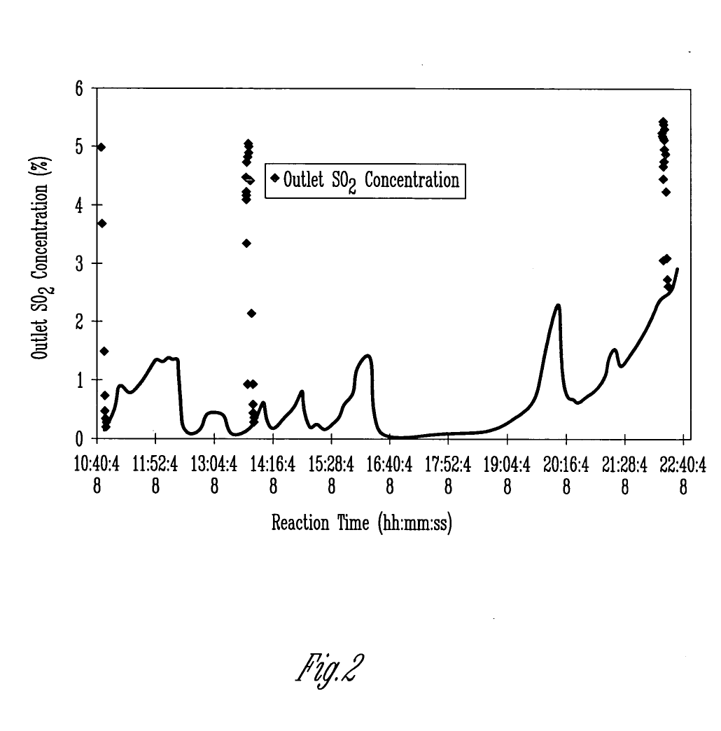

FIG. 2 is a graph illustrating the utilization of SO₂ for the production of acetic acid from calcium acetate generated in corn hull fermentation process.

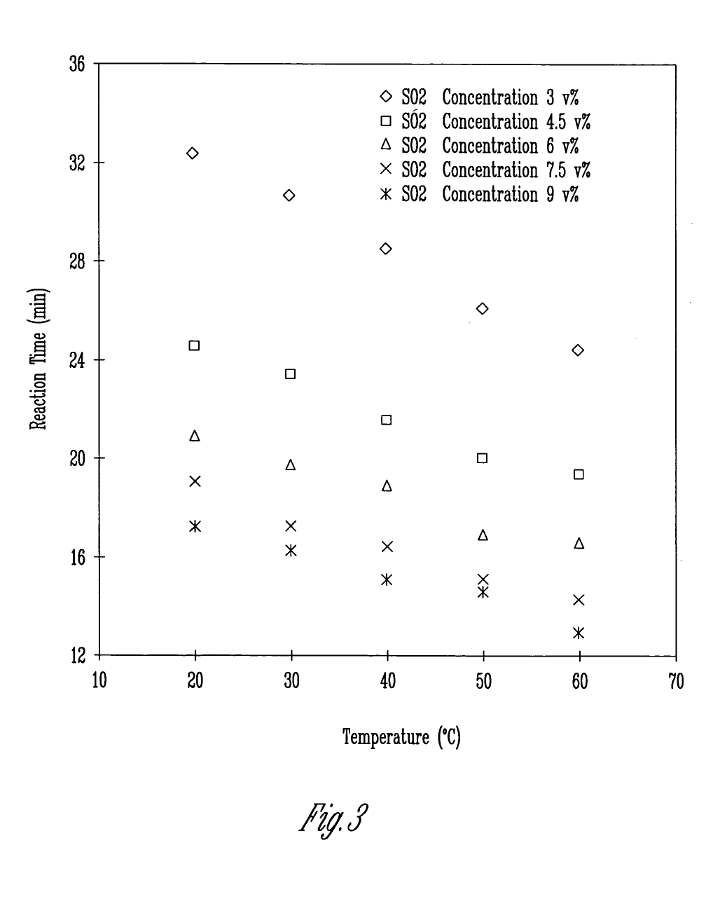

FIG. 3 is a graph illustrating the relationship between the reaction temperature and time needed for the completion of the reaction of sulfur dioxide sparged into calcium acetate solution.

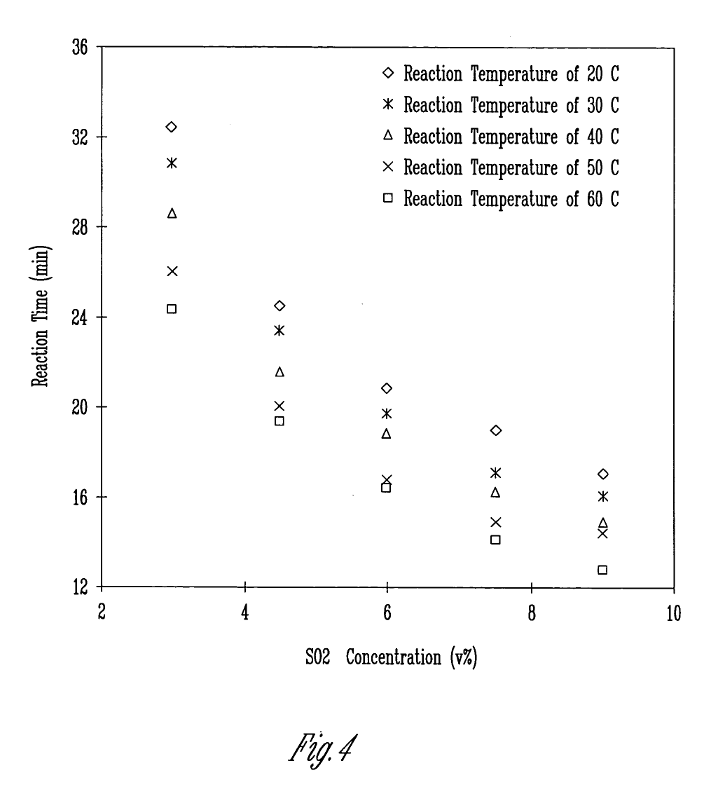

FIG. 4 is a graph illustrating the effect of SO₂ concentration on the rate of reaction of reaction with calcium acetate solution.

### DETAILED DESCRIPTION OF THE INVENTION

Wastewater containing dilute acetic acid is produced in many chemical processes, such as in the manufacture of acetic acid, terephthalic acid, isophthalic acid, cellulose acetate, and trimellitic anhydride. The wastewater in theses processes usually comes from a distillation column which recovers the bulk of acetic acid. The concentration of acetic acid in these wastewaters is very dilute, typically ranging from 0.1 to 5.0 wt %. The wastewaters are further treated by the activated sludge process prior to discharge into the environment.

The most common problems of acetic acid recovery methods involve the separation of a small amount of acetic acid from relatively a large amount of water. Distillation is an energy-intensive method because a large amount of water has to be vaporized. Solvent extraction is a capital-intensive method since a large number of steps are necessary, leading also to low acetic acid recovery efficiency.

The present invention is predicated on the discovery that CaO or Ca(OH)₂ and sulfur oxide may be used to concentrate acetic acid and other carboxylic acid in waste streams. The carboxylic acid may then be used as a raw material for the chemical industry or further concentrated and/or purified using solvent extraction methods or other conventional means.

The process first involves combining calcium oxide (CaO) and/or calcium hydroxide (Ca(OH)₂) with waste water in a continuous flow reactor or other appropriate reaction vessel. The invention may be used to treat any wastewater containing acetic acid or other carboxylic acids including, but not limited to, formic, propionic, butyric, valeric, caproic, enanthic, caprylic, pelargonic, oleic, hexanoic, oxalic, and capric acid. Typical sources of such wastewater include chemical, pharmaceutical, pulp and paper, furfural production, rice and corn milling, fluid milk and cheese production, petrochemical, brewery and meat packing industries, as well as effluents from acidogenic anaerobic fermentation and anaerobic digestion of animal manure. While the methods of the invention are effective in increasing the concentration of carboxylic acid in any waste water, the concentration of carboxylic acid from industrial waste water that is typically used for the invention will generally range from about 0.2-10% by weight, and most frequently in the range of about 0.5-2.5% by weight.

The waste water is combined with any CaO or Ca(OH)₂ source that is effective in neutralizing the carboxylic acid in the waste water, i.e. causing the precipitation of calcium carboxylate. In this respect, the ratio of carboxylic acid in the waste water to calcium oxide will generally range from about 1.07 to 1.50 by weight. CaO and Ca(OH)₂ can be readily obtained through conventional sources.

The neutralization reaction (using acetic acid-containing waste water as an example) can be expressed by reactions (1) and (2):
2CH₃COOH+CaO→Ca(CH₃COO)₂+H₂O (1)
2CH₃COOH+Ca(OH)₂→Ca(CH₃COO)₂+2H₂O (2)

The solubility of the calcium carboxylate is low. Therefore, it will precipitate when its concentration reaches saturation and CaO or Ca(OH)₂ is used in excess. The calcium compound should be reacted with the carboxylic acid-containing waste water for a time period of at least 30 minutes. The precipitated calcium carboxylate can be easily separated from water by conventional means, e.g. precipitation and/or membrane separation, then used to regenerate acetic acid.

Acetic acid regeneration may be carried out in any appropriate reaction vessel, such as a reactor. A preferred apparatus for the regeneration of carboxylic acid in accordance with this invention is shown in FIG. 1. The calcium carboxylate slurry is reacted with SOX, whereby x is 2 or 3. This is accomplished by mixing the calcium carboxylate and excess CaO and/or Ca(OH)₂ with the SOX in the reaction vessel. In one embodiment of the invention, from about 0.1-99.8% by volume SOₓ is used in the reaction, while another embodiment includes about 25-75% by volume. The reaction time depends upon the concentration of SOₓ. Generally speaking, the higher the concentration of SOₓ, the shorter the reaction time. As a general guideline, the SOX should be allowed to react with the calcium carboxylate slurry for a time period of at least 30 minutes.

In one embodiment, the calcium carboxylate slurry is fed continuously into the reactor by a metering pump at a rate of between about 5-35 g/minute. In another embodiment, the calcium carboxylate slurry is fed into the reaction vessel at a rate of about 10-30 g/minute.

In another embodiment of the invention, nitrogen (or air) is used to dilute the SOX so that a larger volume of gas can be bubbled into the reactor, which may benefit the calcium carboxylate (and excess CaO/Ca(OH)₂)/SOₓ reaction by enhancing mass transfer. Rotameters may be used to control the flow rates of N₂ and SOₓ. The concentration of SOₓ in the gas stream may be monitored with an SO₂ analyzer. In one embodiment, the concentration of N₂ in the reaction is between 0-99.9% by weight, with about 25-75% by weight being included in another embodiment.

The temperature of the calcium carboxylate/SOₓ reaction may generally range from about 10-75° C. In one embodiment, the reaction temperature is between about 20-40° C. Ambient temperatures are preferred for purposes of cost and convenience. It should be appreciated, however, that higher temperatures in general result in faster reaction times. Thus, if speed is of the essence, higher reaction temperatures of up to about 70° C. may be preferred.

The reactor temperature can be maintained by such conventional means as a water bath. The pH is not critical, and may generally range between about 2.0-8.0. In one embodiment, the pH ranges between about 2.4-7.9. The reaction time, generally in the range of about 0.5-5 hours, is determined by the quantity of calcium carboxylate added to the reactor, concentration of SOX in the mixed gas stream, and flow rate of the mixed gas stream. The reaction stops when the conversion efficiency of SOₓ at the outlet of gas stream is close to 0. The final concentration of carboxylic acid produced depends on the ratio of calcium carboxylate and water added to the reactor.

The reaction between the SOₓ and calcium carboxylate results in the regeneration of carboxylic acid, which can be described by reactions (3) or (4) (illustrating the regeneration of acetic acid from calcium acetate):
Ca(CH₃COO)₂+SO₂+H₂O→CaSO₃↓+2CH₃COOH (3)
or
Ca(CH₃COO)₂+SO₃+H₂O→CaSO₄↓+2CH₃COOH (4)

The precipitate of calcium sulfite or sulfate can be easily separated by conventional means, such as filtration or precipitation. FIG. 2 demonstrates that SO₂ reacts almost completely with calcium acetate formed by Reaction (1) since most of the SO₂ was removed.

The methods of the invention increase the concentration of carboxylic acid in the carboxylic acid-water mixture several fold. For instance, in one experiment, the inventors were successful in increasing the concentration of acetic acid from 2 wt-% to about 50 wt-% by multiple steps, a 25-fold increase. It is expected that the invention is capable of increasing the concentration of carboxylic acid to even higher levels through continued operation of the reactor. As a practical matter, it is often desired to increase the carboxylic acid concentration of the carboxylic acid-water mixture to at least 25% by weight.

The concentrated carboxylic acid solution obtained by the methods of the invention may be further purified to reach a higher purity of carboxylic acid using conventional methods, such as organic solvent extractions.

Once the reaction process is complete, the separated CaSO₃ or CaSO₄ can be disposed or decomposed for reuse. The calcium sulfite or sulfate generated may be decomposed into SO₂ or SO₃ and CaO as shown by reactions (5) and (6):
CaSO₃→CaO+SO₂ (5)
CaSO₄→CaO+SO₃ (6)

The resulting SO₂ or SO₃ and CaO can be recycled in the overall carboxylic acid recovery process. Thus, the carboxylic acid separation process of the invention does not generate any additional waste product.

The carboxylic acid separation procedure of the present invention has several advantages over conventional distillation processes. First, the invention does not require the use of expensive chemicals. For example, the invention may use inexpensive lime as a source of CaO.

Second, the proposed carboxylic acid separation method can be conducted at a temperature lower than that needed for conventional simple distillation and azeotropic distillation. Thus, the method requires the input of less energy, thereby providing a safer separation environment.

Moreover, the present separation method is supportive of industrial ecology since the CaO and SOₓ can be recycled and used repeatedly. This also decreases the cost of the process.

Most importantly, the present separation method provides faster and better separation efficiency than that obtained by simple distillation and azeotropic distillation.

The following example is offered to illustrate but not limit the invention. Thus, it is presented with the understanding that various formulation modifications as well as method of delivery modifications may be made and still are within the spirit of the invention.

### EXAMPLE 1

### Acetic Acid Regeneration System

The reactor system of this example is shown in FIG. 1. The system consists of a 5 L reactor vessel 20 fitted with a five-port lid. Calcium acetate slurry is fed continuously into reactor 20 using a metering pump 16. Nitrogen from nitrogen tank 10 is used to dilute SOₓ from SOₓ tank 12 so that a larger volume of gas can be bubbled into the reactor 20. The contents of the reactor 20 are mixed using stirrer motor 18.

The concentration of SOₓ in the gas stream is monitored with a SOₓ analyzer 30. The temperature of reactor 20 is maintained by a water bath for which the temperature is controlled by an integrated controller 14. The concentration of SOₓ supplied to the reactor 20 and reactor temperature are automatically recorded with a data acquisition system 32.

Water and acetic acid vapor generated when liquid in reaction vessel 20 is heated can be condensed and returned through condenser 22. Condenser chiller unit 8 may be used to control the temperature of fluid flowing through condenser 22. The stream is passed through a gas dryer 26 (receiving air from air tank 28) before it enters SOX analyzer 30 to eliminate the effect of water and particulate on the measurement of SOX in the outlet gas stream.

### EXAMPLE 2

### Recovery of Acetic Acid with Sulfur Dioxide

Materials and Methods

Materials

Calcium acetate (99.8%) and sulfur dioxide (anhydrous, 99.98%) used in this research was purchased from Fisher Scientific International Inc. and Matheson Tri-Gas Inc. (Montgomeryville, Pa.), respectively.

Apparatus and Operational Procedures

A 500 mL reactor (Chemglass, Inc., Vineland, N.J.) was used to conduct all experiments. Temperature control was realized using a Neslab RTE-111 bath/circulator, which circulated a low-temperature oil (Ace Glass, Inc., Vineland, N.J.) through the jacket of the reactor. To avoid water loss through evaporation, the outlet gas from the reactor passed through a condenser that was maintained at approximately 3° C. by a heated/refrigerated Cole Parmer Polystat® 6-liter circulator unit. The inlet and outlet concentrations of SO₂ in the gas stream were monitored using a California Analytical model ZRF NDIR gas analyzer (manufactured by Fuji Electric Company, Saddle Brook, N.J.). The gas analyzer reads 0 to 10 v % SO₂ by 0.01% and has a repeatability of ±0.5% of full scale. The SO₂ readings of the gas analyzer were recorded with a computer-based data collection system every 10 seconds for further analysis. During the experiments, the reaction mixture in the reactor was stirred at 60 rpm for all trials by an adjustable overhead stirrer connected to a Teflon mixer. Mass measurements of the calcium acetate and water were made on a Mettler model PM4000 balance with a linearity of ±0.02 g. The flow rates of gases were controlled with flow meters. Reaction temperature was measured with a non-mercury glass thermometer inserted into the reaction mixture.

The first step of the reaction was to add 40.0 g Ca(CH₃COO)₂.H₂O into a reactor filled with 245.5 g deionized water and then to stir the mixture continuously at 60 rpm for 30 minutes to completely dissolve all of the added calcium acetate. Since the final concentration of acetic acid generated for all tests was set to be 1.667 M, the quantities of calcium acetate and water added in each test were the same. N₂ and SO₂ were then sparged into the reactor solution through an 8 mm glass tube to start the reaction. The SO₂ gas analyzer was calibrated before and after each test run. The calibrations were performed with known concentrations of standard gases supplied by BOC Gases, Des Moines, Iowa. Each experiment was ended when the outlet concentration of SO₂ was the same as the inlet concentration.

Variables used in this research include reaction temperature and concentration of SO₂ in the gas stream, with a total flow rate of 3447.0 mL/min. The reaction temperature varied from 20 to 60° C., with an interval of 10° C. The concentration of SO₂ in the gas mixture varied from 3.0 to 9.0 v %, with an interval of 1.5 v %.

Analysis of Acetic Acid with HPLC

The acetic acid produced from R1 was analyzed with a Waters 501 high-performance liquid chromatograph (HPLC). The organic acid analysis column used was provided by Alltech Prevail (Alltech Associates, Inc). The material used in mobile phase was a degassed KH₂PO₄ solution (0.005 M). The HPLC operation parameters during the measurements of acetic acid include: 1) 192 nm of UV light, 2) a column pressure of 900 psi, and 3) a mobile phase flow rate of 0.8 mL/min.

Results and Discussion

Once sulfur dioxide was sparged into the calcium acetate solution, it underwent a series of steps before reacting with the calcium acetate, including gas phase diffusion, mass transfer at the gas-liquid interface, hydrolysis and ionization of the dissolved SO₂, and aqueous diffusion and reaction between the calcium acetate and sulfurous acid. The solubility of SO₂ in 100.0 g water is 10.6 g at a temperature of 20° C. and 3.2 g at 60° C., which means that the quantity of SO₂ dissolved in water is considerable given enough time. However, the rate of SO₂ dissolved into water was so slow that the SO₂ concentration difference in the inlet and outlet stream was negligible when only water existing in the reaction vessel. After addition of calcium acetate in the water, the experiment showed that the SO₂ concentrations in the outlet stream remained at zero throughout the process. This suggested that the solution's capacity for absorption of SO₂ was greatly increased by the dissolution of calcium acetate. When SO₂ was dissolved into the solution containing calcium acetate, it reacted to yield HSO₃⁻ and SO₃²⁻, thereby lowering the dissolved SO₂ concentration and allowing more total SO₂ from the gas phase to be dissolved.

The solubility of Ca(CH₃COO)₂ in 100.0 g of water is 37.4 g at a temperature of 0° C. and 29.7 g at 1001C. Under experimental conditions, the added calcium acetate was completely dissolved. The produced calcium sulfite, however, had a very low solubility in water: 0.0043 g at 18° C. and 0.0011 g at 100° C. in 100.0 g water. When SO₂ was sparged into the solution, it dissolved in the water and reacted with calcium acetate to produce calcium sulfite precipitate and acetic acid. Calcium sulfite was separated from the liquid with a simple filtration process.

In the experimental design, the assumption was made that the reaction endpoint would be reached when concentrations of SO₂ in the inlet and outlet mixture gases were identical. At the beginning of the reaction, the outlet gas from the reactor was nondetectable, indicating that the SO₂ in the gas mixture was completely removed by the reaction. At the end of reaction, however, SO₂ was no longer consumed and dissolved into the solution and then the SO₂ concentration of outlet stream started to increase.

Effects of Temperatures on the Reaction Rates

The relationships between reaction temperature and the reaction time needed for the completion of reactions at given reaction conditions are shown in FIG. 1. FIG. 1 shows that the higher the reaction temperature is the shorter the reaction time needed. This fact can be explained with kinetic theory that higher temperature results in a higher reaction rate constant. It can be seen that reaction times at a temperature of 60° C. were about 75% of those at 30° C. In real-world industrial applications with fixed SO₂ flow rates, higher temperatures may yield higher recovery rates-but also a higher levels of energy consumption.

Effects of SO₂ Concentrations on the Reaction Rates

SO₂ concentrations directly affected reaction times. Higher SO₂ concentrations shortened the amount of time needed to complete reactions in the system. FIG. 4 shows that reaction time decreased as the SO₂ flow rate increased—an obvious outcome, since the high SO₂ concentrations represent that more SO₂ was sparged into the system over the same period. However, the reaction time was not proportional to the flow rate at which SO₂ was sparged into the system. The results showed that reaction times under the condition of 3.0 v % of SO₂ concentration were only about 80% greater than those with a concentration of 9.0 v %, assumed previously to be up to 200% greater if SO₂ concentrations determined reaction time. This indicates that reaction in the system was complex, and that the reaction rate was not controlled solely by means of the SO₂ flow rate.

The concentrations of acetic acids produced under different reaction conditions are listed in the Table 1. It shows that SO₂ concentration and reaction temperature had no substantial effect on the concentrations of acetic acid produced. Although there were some deviations from the designed 1.667 M of acetic acid concentration, these differences were random and no indication of effects from these two factors could be found. This result suggests that high concentrations of SO₂ can be used to recover acetic acid from the calcium acetate solution at room temperature. Since reaction at room temperature would save large amounts of the energy needed to heat for reaction, both of these conditions are highly desirable in real-world industrial applications, albeit at the cost of longer reaction times. Increasing SO₂ concentrations, however, can make up this deficiency. Large amount of gas stream containing high concentration of SO₂ is available in new generation of power plants [15, 16], which will make the recovery of acetic acid from biostreams with SO₂ feasible.

TABLE 1
The concentrations of produced acetic acid (M)
based on temperature and SO₂ concentration

| | Temperature (° C.) | | | | |
|---|---|---|---|---|---|
| SO₂ Concentration (v %) | 20 | 30 | 40 | 50 | 60 |
| 3.0 | 1.636 ± | 1.561 ± | 1.606 ± | 1.671 ± | 1.629 ± |
| | 0.030 | 0.017 | 0.024 | 0.012 | 0.026 |
| 4.5 | 1.666 ± | 1.721 ± | 1.624 ± | 1.702 ± | 1.658 ± |
| | 0.012 | 0.029 | 0.049 | 0.029 | 0.036 |
| 6.0 | 1.669 ± | 1.650 ± | 1.661 ± | 1.665 ± | 1.666 ± |
| | 0.050 | 0.053 | 0.055 | 0.035 | 0.008 |
| 7.5 | 1.658 ± | 1.692 ± | 1.679 ± | 1.658 ± | 1.647 ± |
| | 0.013 | 0.029 | 0.063 | 0.013 | 0.012 |
| 9.0 | 1.673 ± | 1.666 ± | 1.645 ± | 1.695 ± | 1.571 ± |
| | 0.009 | 0.023 | 0.010 | 0.038 | 0.003 |

Summary

Sulfur dioxide can be used to recover acetic acid efficiently from calcium acetate solutions. The experimental results show that the time required for a complete reaction decreases with an increase of reaction temperature and SO₂ flow rate. Although a change of reaction conditions leads to a change of reaction time, analysis of the produced acetic acid concentrations demonstrates that the complete conversion of calcium acetate to acetic acid was not affected. This suggests that the recovery process can be designed using a higher SO₂ flow rate at room temperature without affecting recovery efficiency. Since energy for heating is substantially reduced, the latter feature is economically attractive for the industrial recovery of acetic acid from biological fermentation broth. Industry can either increase the flow rate of SO₂ containing gas or even use pure SO₂ gas.

For the above-stated reasons, it is submitted that the present invention accomplishes at least all of its stated objectives.

Having described the invention with reference to particular compositions and methods, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

1. A method of recovering carboxylic acid comprising:
mixing a source of calcium carboxylate with SOₓ to form carboxylic acid and CaSO_{y},
whereby x is 2 or 3, and y is 3 or 4.

2. The method of claim 1 whereby the source of calcium carboxylate and the SOX are combined at a temperature ranging from about 10-75° C.

3. The method of claim 2 whereby the source of calcium carboxylate and the SOₓ are combined at a temperature ranging from about 20-40° C.

4. The method of claim 1 whereby the SOₓ is mixed with the calcium carboxylate in a concentration of up to about 10 v %.

5. The method of claim 4 whereby the SOX is mixed with the calcium carboxylate in a concentration of between about 3-9 v %.

6. The method of claim 1 whereby the SOₓ is mixed with the calcium carboxylate by adding a constant flow of SOₓ.

7. The method of claim 1 further including the step of forming the source of calcium carboxylate by combining carboxylic acid-containing waste water with a calcium compound.

8. The method of claim 7 whereby the calcium compound is selected from the group consisting of calcium oxide, calcium hydroxide, and combinations thereof.

9. A method of recovering carboxylic acid from byproduct or waste streams comprising:
combining carboxylic acid-containing waste water with a calcium compound to produce calcium carboxylate; and mixing the calcium carboxylate with SOₓ to form regenerated carboxylic acid and CaSO_{y}, whereby x is 2 or 3, and y is 3 or 4.

10. The method of claim 9 whereby the calcium compound is selected from the group consisting of calcium oxide, calcium hydroxide, and combinations thereof.

11. The method of claim 9 whereby the ratio of carboxylic acid in waste water to the calcium compound is about 1.07-1.50 by weight.

12. The method of claim 9 whereby the waste water has an initial concentration of carboxylic acid of between about 0.2-10% by weight.

13. The method of claim 9 whereby the waste water has a concentration of regenerated carboxylic acid of at least 25% by weight.

14. The method of claim 9 whereby the calcium carboxylate is combined with about 25-75% by volume SOX.

15. The method of claim 9 further including the step of diluting the SOX with nitrogen prior to mixing the SOX with the calcium carboxylate.

16. The method of claim 15 whereby the nitrogen is included in a concentration of from about 25-75% by volume.

17. The method of claim 9 whereby the combining and the mixing steps take place at a temperature in the range of between about 10-75° C.

18. The method of claim 17 whereby the combining and the mixing steps take place at a temperature in the range of between about 20-40° C.

19. The method of claim 9 whereby the waste water and the calcium oxide are allowed to react for a time period of at least 30 minutes.

20. The method of claim 9 whereby the calcium carboxylate and the SOX are allowed to react for a time period of at least 30 minutes.

21. The method of claim 9 further including the step of decomposing the CaSO_{y} to form calcium oxide and SOₓ.

22. The method of claim 21 whereby the calcium oxide is recycled and used in the combining step.

23. The method of claim 21 whereby the SOX is recycled and used in the mixing step.

24. The method of claim 9 whereby the combining and the mixing steps occur at a pH of between about 2.0-8.0.

25. The method of claim 9 further including the step of purifying the regenerated carboxylic acid.

26. The method of claim 9 whereby the SOX is mixed with the calcium carboxylate in a concentration of up to about 10 v %.

27. The method of claim 26 whereby the SOX is mixed with the calcium carboxylate in a concentration of between about 3-9 v %.

28. The method of claim 9 whereby the SOX is mixed with the calcium carboxylate by adding a constant flow of SOX.
